(19) United States
(12) Patent Application Publication
(10) Pub. No.: US 2009/0186344 A1
(43) Pub. Date: Jul. 23, 2009

(54) DEVICES AND METHODS FOR DETECTING AND QUANTITATING NUCLEIC ACIDS USING SIZE SEPARATION OF AMPLICONS

(75) Inventor: Javier A. Farinas, Los Altos, CA (US)

Correspondence Address: CARDINAL LAW GROUP;Caliper Life Sciences, Inc., 1603 Orrington Avenue, Suite 2000, Evanston, IL, 60201, US

(73) Assignee: CALIPER LIFE SCIENCES, INC., Mountain View, CA (US)

(21) Appl. No.: 12/056,213

(22) Filed: Mar. 26, 2008

Related U.S. Application Data
(60) Provisional application No. 61/062,059, filed on Jan. 23, 2008.

(51) Int. Cl. C12Q 1/68

(52) U.S. Cl. 435/6

(57) ABSTRACT

Devices and methods are described for detecting and quantifying nucleic acids using a sealed system that minimizes contamination. In particular, provided herein are devices for and methods using nucleic acid amplification that permit multiple sampling of an amplification reaction mixture and quantitation and identification of amplicons during the course of an amplification reaction. Methods involving the transfer of samples from an amplification reaction mixture into a separation network, separation of nucleic acids based on size, and identification and quantitation of nucleic acids are disclosed.

### CROSS-REFERENCE TO RELATED APPLICATIONS

The present application claims priority to U.S. Provisional Patent Application Ser. No. 61/062,059, filed Jan. 23, 2008, entitled “Devices and Methods for Detecting and Quantitating Nucleic Acids using Size-Separation of Amplicons,” which is hereby incorporated by reference for all purposes as if set forth herein verbatim.

### BACKGROUND OF THE INVENTION

1. Field of the Invention

The present disclosure is in the field of nucleic acid detection. In particular, described herein are devices and methods for detecting and quantitating nucleic acids using a sealed system that minimizes contamination.

2. Description of the Related Art

Detection of nucleic acids is central to gene expression analysis, diagnostics, medicine, forensics, industrial processing, crop and animal breeding, and many other fields. For example, nucleic acid detection technology is used to diagnose disease conditions, detect infectious organisms, determine genetic lineage and genetic markers, correctly identify individuals at crime scenes, and propagate industrial organisms.

The introduction of nucleic acid amplification methods has greatly improved the specificity and sensitivity of nucleic acid detection. One of the most commonly used methods of nucleic acid amplification is polymerase chain reaction (PCR), which amplifies nucleic acids by using sequence specific primers targeted to opposing strands of double stranded DNA to copy a desired DNA sequence. Multiple cycles of primer annealing, DNA polymerization and double-stranded DNA denaturation are used to exponentially amplify a desired segment of DNA. Reactions with only one copy of template DNA can be rapidly and specifically amplified more than 100 million fold (see, e.g., U.S. Pat. Nos. 4,683,195, 4,683,202 and 4,800,159).

Other methods for amplification of nucleic acids include reverse-transcriptase PCR (RT-PCR), nucleic acid sequence-based amplification (NASBA), transcription-based amplification system (TAS), self-sustained sequence replication (3SR), ligation amplification reaction (LAR), Q-beta amplification, and ligase chain reaction (LCR). Many of these amplification reactions utilize a polymerase enzyme or fragment of such an enzyme.

Details regarding the use of these and other amplification methods can be found in any of a variety of standard texts, including, e.g., Sambrook et al., Molecular Cloning—A Laboratory Manual (3rd Ed.), Vol. 1-3, Cold Spring Harbor Laboratory, Cold Spring Harbor, N.Y., 2000 (“Sambrook”); Current Protocols in Molecular Biology, F. M. Ausubel et al., eds., Current Protocols, a joint venture between Greene Publishing Associates, Inc. and John Wiley & Sons, Inc., (supplemented through 2002) (“Ausubel”) and PCR Protocols A Guide to Methods and Applications (Innis et al. eds) Academic Press Inc. San Diego, Calif. (1990) (Innis). Many available biology texts have extended discussions regarding PCR and related amplification methods.

There are many methods for detecting amplified nucleic acid products. Some methods (see, e.g., U.S. Pat. No. 4,683,195) utilize dot-blots, oligonucleotide arrays, size-separation by gel electrophoresis, Sanger sequencing, and various hybridization probes, and may require post-reaction processing. For in situ detection of amplification products during the amplification reaction, intercalating dyes (see, e.g., U.S. Pat. Nos. 5,994,056; 6,171,785 and 6,814,934), TaqMan (U.S. Pat. No. 5,210,015) and Scorpion probes are commonly used.

More recently, a number of minituarized approaches to performing PCR and other amplification reactions have been developed, e.g., involving amplification reactions in microfluidic devices, as well as methods for detecting and analyzing amplified nucleic acids in or on the devices. Details regarding such technology can be found in the technical and patent literature (e.g., Kopp et al. (1998) “Chemical Amplification: Continuous Flow PCR on a Chip” Science, 280 (5366):1046; U.S. Pat. No. 6,444,461 to Knapp, et al. (Sep. 3, 2002) MICROFLUIDIC DEVICES AND METHODS FOR SEPARATION; U.S. Pat. No. 6,406,893 to Knapp, et al. (Jun. 18, 2002) MICROFLUIDIC METHODS FOR NON-THERMAL NUCLEIC ACID MANIPULATIONS; U.S. Pat. No. 6,391,622 to Knapp, et al. (May 21, 2002) CLOSED-LOOP BIOCHEMICAL ANALYZERS; U.S. Pat. No. 6,306,590 to Mehta, et al. (Oct. 23, 2001) MICROFLUIDIC MATRIX LOCALIZATION APPARATUS AND METHODS; U.S. Pat. No. 6,303,343 to Kopf-Sill (Oct. 16, 2001) INEFFICIENT FAST PCR; U.S. Pat. No. 6,171,850 to Nagle, et al. (Jan. 9, 2001) INTEGRATED DEVICES AND SYSTEMS FOR PERFORMING TEMPERATURE CONTROLLED REACTIONS AND ANALYSES; U.S. Pat. No. 5,939,291 to Loewy, et al. (Aug. 17, 1999) MICROFLUIDIC METHOD FOR NUCLEIC ACID AMPLIFICATION; U.S. Pat. No. 5,955,029 to Wilding, et al. (Sep. 21, 1999) MESOSCALE POLYNUCLEOTIDE AMPLIFICATION DEVICE AND METHOD; U.S. Pat. No. 5,965,410 to Chow, et al. (Oct. 12, 1999) ELECTRICAL CURRENT FOR CONTROLLING FLUID PARAMETERS IN MICROCHANNELS, and many others).

Despite the wide-spread use of amplification technologies and the adaptation of these technologies to minituarized systems, certain technical difficulties persist in amplifying and detecting nucleic acids. Nucleic acid amplification methods, because of their ability to greatly amplify template nucleic acids, are prone to false positive results due to mis-annealing of primers or sample contamination, particularly contamination from previously amplified nucleic acids. While methods exist for simultaneous amplification and detection of nucleic acids, these methods are limited either by the need to sample an open vessel and thus suffer from contamination concerns, or by the inability to distinguish different optical signals, which limits the level of multiplex analysis that can be carried out. Although methods have been described previously for integrating PCR and size separation of nucleic acids (Anal. Chem. 2001:73:565-570), it would be desireable to develop methods for multiple sampling of a PCR chamber to allow for quantitation and identification of amplicons during the course of an amplification reaction.

Thus, there remains a need for improved methods for detecting and quantifying nucleic acids that permit multiplex analysis with increased accuracy while minimizing contamination.

### BRIEF SUMMARY OF THE INVENTION

In one aspect, provided herein is a method for detecting and quantifying one or more specific nucleic acids in a sealed system that limits contamination. In one embodiment, the method comprises: a) providing a device comprising i) a reaction chamber containing a reaction mixture comprising one or more nucleic acids and reagents for specific nucleic acid amplification, ii) a separation network, and iii) a fluidic connection between the reaction chamber and the separation network, wherein an aliquot of the reaction mixture can be introduced from the reaction chamber into the separation network; b) sealing the reaction chamber and network so that the nucleic acids cannot be transferred out of the device; c) processing the reaction mixture in the reaction chamber whereby one or more nucleic acids are amplified, d) periodically transferring an aliquot of the reaction mixture from the reaction chamber to the separation network during the amplification reaction of the nucleic acids; e) separating the nucleic acids in the sample based on the size of the nucleic acids; and f) detecting the separated nucleic acids. Because the system is sealed, it can be discarded after one use without risk of contaminating future amplification reactions. In certain embodiments, the sample comprises more than one nucleic acid, for example, at least 2, 3, 4, 5, 6, 7, 8, 9, 10, 50 or more nucleic acids.

Nucleic acids can be amplified by PCR or any other method for nucleic acid amplification, such as but not limited to, reverse-transcriptase PCR (RT-PCR), nucleic acid sequence-based amplification (NASBA), transcription-based amplification system (TAS), self-sustained sequence replication (3SR), ligation amplification reaction (LAR), Q-beta amplification, and ligase chain reaction (LCR).

The reaction chamber where nucleic acid amplification takes place is fluidically connected to a separation network such that the whole system is sealed so that reagents under normal operating conditions cannot leave the system. In certain embodiments, a potential difference such as a pressure difference or a voltage difference is used to transfer an aliquot comprising one or more nucleic acids from the reaction chamber to the separation network.

The separation network comprises channels, tubing, wells, or some combination thereof that can be used to separate nucleic acids or fragments thereof based on size. Exemplary separation networks include microfluidic networks, capillary electrophoresis (CE) capillaries, high performance liquid chromatography (HPLC) columns, and the like. In certain embodiments, the separation network comprises a sieving matrix comprising a polymer selected from the group consisting of linear acrylamide, polyacrylamide, polydimethylacrylamide, polydimethylacrylamide/coacrylic acid, agarose, methyl cellulose, polyethylene oxide, hydroxycellulose, and hydroxy ethyl cellulose.

After addition of the amplification reaction mixture to the reaction chamber and filling of the separation network with an appropriate separation matrix, the system is sealed. Such sealing can be done for example with a film or membrane. The sealing needs to be done in a manner that the required driving forces can be applied to the separation network and reaction chamber to allow for sampling of the reaction chamber and size separation of the sample. For example, the sealing can be done with a compliant membrane that prevents liquid or aerosols from leaving the device but allows pressure differentials to be applied between the reaction chamber and the separation network. In the case of electrophoretic separations, electrodes can be embedded into the device in such a way that an instrument can control voltages in the device via the embedded electrocodes, eliminating the need to place external eletrodes into contact with the fluid inside the device. In one embodiment, the system comprises a microfluidic device for gel electrophoresis.

Nucleic acids can be detected by a variety of means, including, but not limited to, measuring the absorbance of a nucleic acid or detecting labeled reagents, such as labeled oligonucleotide primers or fluorescent intercalating dyes (e.g., ethidium bromide, SYBR green, SYTO-9, SYTO-13, SYTO-16, SYTO-60, SYTO-62, SYTO-64, SYTO-82, PO-PRO-3, YO-PRO-1, SYTOX Orange, and TO-PRO-3). The particular optical signal that is used for measurements depends on the size and concentration of the amplicon. In certain embodiments, the nucleic acids are detected with a detector comprising a fluorometer, a charge coupled device, a laser, an enzyme, an enzyme substrate, a photo multiplier tube, a spectrophotometer, scanning detector, microscope, or a galvo-scanner. Nucleic acids can be measured at time points before, during, and after the amplification reaction.

The amplicons formed in the reaction chamber are measured outside of the reaction chamber while maintaining a sealed system. For example, nucleic acids can be detected and quantified by making an optical measurement in the separation network that is related to the size and concentration of the specific nucleic acids in the sample. Inclusion of reagents for detection of nucleic acids within the separation network, and not the reaction chamber, has the advantage of not requiring optimization of the amplification reaction in the presence of the dye. Separation of the amplification and detection functions allows for optimization of reaction and detection conditions independently.

In another aspect, provided herein is a sealed system for detecting nucleic acids comprising a) a reaction chamber containing a reaction mixture comprising a sample comprising one or more nucleic acids and reagents for specific nucleic acid amplification, b) a separation network, and c) a fluidic connection between the reaction chamber and the separation network, wherein an aliquot of the reaction mixture can be introduced from the chamber into the separation network. In certain embodiments, the separation network comprises a microfluidic network, a CE capillary, an HPLC column, or any combination thereof. In certain embodiments, the separation network comprises a sieving matrix comprising a polymer selected from the group consisting of linear acrylamide, polyacrylamide, polydimethylacrylamide, polydimethylacrylamide/coacrylic acid, agarose, methyl cellulose, polyethylene oxide, hydroxycellulose, and hydroxy ethyl cellulose. In certain embodiments, the system is a miniaturized system comprising a microfluidic device. In one embodiment, the system comprises a microfluidic device for gel electrophoresis. In certain embodiments, the system further comprises a detector, such as but not limited to, a fluorometer, a charge coupled device, a laser, an enzyme, an enzyme substrate, a photo multiplier tube, a spectrophotometer, scanning detector, microscope, or a galvo-scanner.

In another aspect, the invention includes a kit comprising a system for detecting nucleic acids, as described herein, and instructions for detecting and quantifying nucleic acids.

Thus, the present disclosure encompasses, but is not limited to, the following numbered embodiments:

1. A method for detecting and quantifying one or more specific nucleic acids in a sealed system, the method comprising:
a) providing a device comprising:
   i) a reaction chamber containing a reaction mixture comprising one or more nucleic acids and reagents for specific nucleic acid amplification,
   ii) a separation network, and
   iii) a fluidic connection between the reaction chamber and the separation network, wherein an aliquot of the reaction mixture can be removed from the chamber into the separation network;
b) sealing the reaction chamber and network so that the nucleic acids cannot be transferred out of the device;
c) processing the reaction mixture in the reaction chamber whereby one or more nucleic acids are amplified,
d) periodically transferring an aliquot of the reaction mixture from the reaction chamber to the separation network during the amplification reaction of the nucleic acids;
e) separating the nucleic acids in the separation network based on the size of the nucleic acids; and
f) detecting the separated nucleic acids.

2. The method of embodiment 1, wherein the nucleic acids are amplified by a method selected from the group consisting of polymerase chain reaction (PCR), reverse-transcriptase PCR (RT-PCR), nucleic acid sequence-based amplification (NASBA), transcription-based amplification system (TAS), self-sustained sequence replication (3SR), ligation amplification reaction (LAR), Q-beta amplification, and ligase chain reaction (LCR).

3. The method of embodiment 1, wherein samples are withdrawn from the reaction chamber after each cycle of nucleic acid amplification by applying a pressure between the reaction chamber and the separation network.

4. The method of embodiment 1, wherein more than one nucleic acid is analyzed.

5. The method of embodiment 4, wherein at least 7 nucleic acid templates are analyzed.

6. The method of embodiment 1, wherein the separation network comprises channels, tubing, or wells that can be used to separate nucleic acids or fragments thereof based on size.

7. The method of embodiment 6, wherein the separation network comprises a microfluidic network.

8. The method of embodiment 1, wherein the nucleic acids are separated electrophoretically.

9. The method of embodiment 8, wherein the separation network comprises a CE capillary.

10. The method of embodiment 1, wherein the nucleic acids are separated chromatographically.

11. The method of embodiment 10, wherein the separation network comprises an HPLC column.

12. The method of embodiment 1, wherein the nucleic acids are separated by flowing said nucleic acids through a sieving matrix.

13. The method of embodiment 12, wherein the separation network comprises a sieving matrix comprising a polymer selected from the group consisting of linear acrylamide, polyacrylamide, polydimethylacrylamide, polydimethylacrylamide/coacrylic acid, agarose, methyl cellulose, polyethylene oxide, hydroxycellulose, and hydroxy ethyl cellulose.

14. The method of embodiment 1, wherein the reaction chamber is sealed with a film.

15. The method of embodiment 1, wherein the reaction chamber is sealed with a membrane.

16. The method of embodiment 15, wherein the membrane is a compliant membrane that prevents liquid or aerosols from leaving the device, but allows pressure differentials to be applied between the reaction chamber and the separation network.

17. The method of embodiment 1, wherein the separation network comprises an electrophoresis device.

18. The method of embodiment 17, wherein the system comprises a microfluidic device for gel electrophoresis.

19. The method of embodiment 1, wherein the nucleic acids are detected with a detector comprising a fluorometer, a charge coupled device, a laser, an enzyme, an enzyme substrate, a photo multiplier tube, a spectrophotometer, scanning detector, microscope, or a galvo-scanner.

20. The method of embodiment 19, wherein the nucleic acids are detected by measuring absorbance.

21. The method of embodiment 19, wherein the nucleic acids are detected by measuring fluorescence.

22. The method of embodiment 1, wherein the nucleic acids are detected by measuring one or more signals from one or more detectably labeled probes that selectively bind to the nucleic acids.

23. The method of embodiment 1, wherein the nucleic acids are detected by measuring one or more signals from one or more detectably labeled primers incorporated into the nucleic acids during amplification.

24. The method of embodiment 1, wherein the nucleic acids are detecting by measuring the signal from an intercalating dye.

25. The method of embodiment 24, wherein the intercalating dye is ethidium bromide or SYBR green.

26. The method of embodiment 1, wherein reagents for detection of nucleic acids are added to the separation network.

27. The method of embodiment 1, wherein nucleic acids are detected by measurement of fluorescence from an intercalating dye in the separation network.

28. The method of embodiment 1, wherein a nucleic acid of interest in the sample is quantified by amplifying the nucleic acid of interest through a plurality of amplification cycles; detecting signals associated with amplicons produced for two or more of the amplification cycles; preparing a sample curve of a signal parameter versus a number of amplification cycles; and, comparing one or more identifiable points from the sample curve to a standard curve of identifiable points versus concentration, thereby quantifying the nucleic acid of interest.

29. The method of embodiment 1, wherein the nucleic acids are amplified by PCR.

30. The method of embodiment 29, wherein samples are withdrawn from the reaction chamber during each thermocycle by applying a pressure between the reaction chamber and the separation network, separation of nucleic acids is based on microfluidic gel electrophoresis, and detection of nucleic acids is accomplished by fluorescence of an intercalating dye introduced in the separation network.

31. The method of embodiment 30, wherein the intercalating dye is ethidium bromide.

32. The method of embodiment 30, wherein the intercalating dye is SYBR green.

33. A sealed device comprising:
a) a reaction chamber containing a reaction mixture comprising a sample comprising one or more nucleic acids and reagents for nucleic acid amplification,
b) a separation network, and
c) a fluidic connection between the reaction chamber and the separation network, wherein a sample of the reaction mixture can be introduced from the reaction chamber into the separation network.

34. The device of embodiment 33, wherein the device is a microfluidic device for gel electrophoresis.

35. The device of embodiment 33, wherein samples are withdrawn from the reaction chamber by applying a pressure between the reaction chamber and the separation network.

36. The device of embodiment 33, further comprising a detector, wherein said detector comprises a fluorometer, a charge coupled device, a laser, an enzyme, an enzyme substrate, a photo multiplier tube, a spectrophotometer, scanning detector, microscope, or a galvo-scanner.

37. The device of embodiment 33, wherein the separation network comprises a CE capillary.

38. The device of embodiment 33, wherein the separation network comprises an HPLC column.

39. The device of embodiment 33, wherein the separation network comprises a sieving matrix.

40. The device of embodiment 39, wherein the separation network comprises a sieving matrix comprising a polymer selected from the group consisting of linear acrylamide, polyacrylamide, polydimethylacrylamide, polydimethylacrylamide/coacrylic acid, agarose, methyl cellulose, polyethylene oxide, hydroxycellulose, and hydroxy ethyl cellulose.

41. The device of embodiment 33, wherein the reaction chamber is sealed with a film.

42. The device of embodiment 33, wherein the reaction chamber is sealed with a membrane.

43. A kit comprising the device of embodiment 33 and instructions for detecting and quantifying nucleic acids.

These and other embodiments will readily occur to those of skill in the art in view of the disclosure herein.

### BRIEF DESCRIPTION OF THE FIGURES

FIG. 1 shows a schematic representation of an exemplary device, as described herein. Shown are a PCR reaction chamber 10 connected to a separation network 20. Wells 30 in the separation network provide means to add reagents to the network and apply pressure and electrical gradients.

FIG. 2 shows bands on a LabChip® 90 microfluidics system that was loaded with nucleic acid samples serially separated during the course of 30 cycles of PCR using a device as described in Example 1. Major bands are from a DNA ladder included on the chip as a reference. The amplicon is clearly visible as a band of increasing intensity at about 200 bp.

FIG. 3 shows detection of the 200 bp amplicon of FIG. 2 during the exponential and plateau phases of the PCR reaction.

### DETAILED DESCRIPTION OF INVENTION

The practice of the present invention will employ, unless otherwise indicated, conventional methods of chemistry, biochemistry, and recombinant DNA techniques, within the skill of the art. Such techniques are explained fully in the literature. See, e.g., A. L. Lehninger, Biochemistry (Worth Publishers, Inc., current addition); Sambrook, et al., Molecular Cloning: A Laboratory Manual (2nd Edition, 1989); Short Protocols in Molecular Biology, 4th ed. (Ausubel et al. eds., 1999, John Wiley & Sons); Molecular Biology Techniques: An Intensive Laboratory Course, (Ream et al., eds., 1998, Academic Press); PCR (Introduction to Biotechniques Series), 2nd ed. (Newton & Graham eds., 1997, Springer Verlag); and Methods In Enzymology (S. Colowick and N. Kaplan eds., Academic Press, Inc.).

All publications, patents and patent applications cited herein, whether supra or infra, are hereby incorporated by reference in their entireties.

### DEFINITIONS

In describing the present invention, the following terms will be employed, and are intended to be defined as indicated below.

It must be noted that, as used in this specification and the appended claims, the singular forms “a”, “an” and “the” include plural referents unless the content clearly dictates otherwise. Thus, for example, reference to “a nucleic acid” includes a mixture of two or more such nucleic acids, and the like.

An “aliquot” is a portion of a component of interest (e.g., a sample or reaction mixture). The aliquot can be diluted, concentrated or undiluted as compared to the component of interest.

The terms “polynucleotide,” “oligonucleotide,” “nucleic acid” and “nucleic acid molecule” are used herein to include a polymeric form of nucleotides of any length, either ribonucleotides or deoxyribonucleotides. This term refers only to the primary structure of the molecule. Thus, the term includes triple-, double- and single-stranded DNA, as well as triple-, double- and single-stranded RNA. It also includes modifications, such as by methylation and/or by capping, and unmodified forms of the polynucleotide. More particularly, the terms “polynucleotide,” “oligonucleotide,” “nucleic acid” and “nucleic acid molecule” include polydeoxyribonucleotides (containing 2-deoxy-D-ribose), polyribonucleotides (containing D-ribose), any other type of polynucleotide which is an N— or C-glycoside of a purine or pyrimidine base, and other polymers containing nonnucleotidic backbones, for example, polyamide (e.g., peptide nucleic acids (PNAs)) and polymorpholino (commercially available from the Anti-Virals, Inc., Corvallis, Oreg., as Neugene) polymers, and other synthetic sequence-specific nucleic acid polymers providing that the polymers contain nucleobases in a configuration which allows for base pairing and base stacking, such as is found in DNA and RNA. There is no intended distinction in length between the terms “polynucleotide,” “oligonucleotide,” “nucleic acid” and “nucleic acid molecule,” and these terms will be used interchangeably. Thus, these terms include, for example, 3′-deoxy-2′,5′-DNA, oligodeoxyribonucleotide N3′ P5′ phosphoramidates, 2′-O-alkyl-substituted RNA, double- and single-stranded DNA, as well as double- and single-stranded RNA, DNA:RNA hybrids, and hybrids between PNAs and DNA or RNA, and also include known types of modifications, for example, labels which are known in the art, methylation, “caps,” substitution of one or more of the naturally occurring nucleotides with an analog, internucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalklyphosphoramidates, aminoalkylphosphotriesters), those containing pendant moieties, such as, for example, proteins (including nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide or oligonucleotide. In particular, DNA is deoxyribonucleic acid.

A “nucleic acid of interest” is any nucleic acid to be amplified, detected and/or quantified in a sample. A nucleic acid of interest can be detected and identified in fragmented form and/or in unfragmented form using methods and systems described herein.

A “template molecule” refers to a molecule of specific identity which can serve as a template for the synthesis of a complementary molecule. Most often, a “template molecule” is a polymeric molecule. In preferred embodiments, a “template molecule” is a nucleic acid, e.g., DNA, RNA, a nucleic acid comprising both deoxyribo- and ribonucleotides, or a nucleic acids comprising deoxyribonucleotides, ribonucleotides, and/or analogs and derivatives thereof. In the context of PCR, a “template molecule” may represent a fragment or fraction of the nucleic acids added to the reaction. Specifically, a “template molecule” refers to the sequence between and including the two primers.

An “amplification reaction” is a reaction that 1) results in amplification of a template, or 2) would result in amplification of a template if the template were present. Thus, an “amplification reaction” can be performed on a sample aliquot that comprises a nucleic acid to be amplified, or on a sample aliquot that does not comprise the nucleic acid. Actual amplification of a template is not a requirement for performing an amplification reaction.

As used herein, a “reaction mixture” refers to a mixture of constituents of an amplification reaction and/or a hybridization reaction. An aliquot of a reaction mixture containing a nucleic acid of interest, or not, can still be considered a reaction mixture.

A nucleic acid is “quantified” or “quantitated” in a sample when an absolute or relative amount of the nucleic acid in a sample is determined. This can be expressed as a number of copies, a concentration of the nucleic acid, a ratio or proportion of the nucleic acid to some other constituent of the sample (e.g., another nucleic acid), or any other appropriate expression.

“Substantially purified” generally refers to isolation of a substance (compound, polynucleotide, oligonucleotide, nucleic acid composition) such that the substance comprises the majority percent of the sample in which it resides. Typically in a sample, a substantially purified component comprises 50%, preferably 80%-85%, more preferably 90-95% of the sample. Techniques for purifying polynucleotides of interest are well-known in the art and include, for example, ion-exchange chromatography, affinity chromatography and sedimentation according to density.

By “isolated” is meant, when referring to a polynucleotide, a nucleic acid molecule devoid, in whole or part, of sequences normally associated with it in nature; or a sequence, as it exists in nature, but having heterologous sequences in association therewith; or a molecule disassociated from the chromosome.

A “DNA-dependent DNA polymerase” is an enzyme that synthesizes a complementary DNA copy from a DNA template. Examples are DNA polymerase I from E. coli and bacteriophage T7 DNA polymerase. All known DNA-dependent DNA polymerases require a complementary primer to initiate synthesis. Under suitable conditions, a DNA-dependent DNA polymerase may synthesize a complementary DNA copy from an RNA template.

A “DNA-dependent RNA polymerase” or a “transcriptase” is an enzyme that synthesizes multiple RNA copies from a double-stranded or partially-double stranded DNA molecule having a (usually double-stranded) promoter sequence. The RNA molecules (“transcripts”) are synthesized in the 5′ to 3′ direction beginning at a specific position just downstream of the promoter. Examples of transcriptases are the DNA-dependent RNA polymerase from E. coli and bacteriophages T7, T3, and SP6.

An “RNA-dependent DNA polymerase” or “reverse transcriptase” is an enzyme that synthesizes a complementary DNA copy from an RNA template. All known reverse transcriptases also have the ability to make a complementary DNA copy from a DNA template; thus, they are both RNA- and DNA-dependent DNA polymerases. A primer is required to initiate synthesis with both RNA and DNA templates.

“As used herein, the term “target nucleic acid region” or “target nucleic acid” denotes a nucleic acid molecule with a “target sequence” to be amplified. The target nucleic acid may be either single-stranded or double-stranded and may include other sequences besides the target sequence, which may not be amplified. The term “target sequence” refers to the particular nucleotide sequence of the target nucleic acid which is to be amplified. The target sequence may include a probe-hybridizing region contained within the target molecule with which a probe will form a stable hybrid under desired conditions. The “target sequence” may also include the complexing sequences to which the oligonucleotide primers complex and extended using the target sequence as a template. Where the target nucleic acid is originally single-stranded, the term “target sequence” also refers to the sequence complementary to the “target sequence” as present in the target nucleic acid. If the “target nucleic acid” is originally double-stranded, the term “target sequence” refers to both the plus (+) and minus (−) strands (or sense and anti-sense strands).

The term “primer” or “oligonucleotide primer” as used herein, refers to an oligonucleotide that hybridizes to the template strand of a nucleic acid and initiates synthesis of a nucleic acid strand complementary to the template strand when placed under conditions in which synthesis of a primer extension product is induced, i.e., in the presence of nucleotides and a polymerization-inducing agent such as a DNA or RNA polymerase and at suitable temperature, pH, metal concentration, and salt concentration. The primer is preferably single-stranded for maximum efficiency in amplification, but may alternatively be double-stranded. If double-stranded, the primer can first be treated to separate its strands before being used to prepare extension products. This denaturation step is typically effected by heat, but may alternatively be carried out using alkali, followed by neutralization. Thus, a “primer” is complementary to a template, and complexes by hydrogen bonding or hybridization with the template to give a primer/template complex for initiation of synthesis by a polymerase, which is extended by the addition of covalently bonded bases linked at its 3′ end complementary to the template in the process of DNA or RNA synthesis.

The term “amplicon” refers to the amplified nucleic acid product of a PCR reaction or other nucleic acid amplification process (e.g., reverse-transcriptase PCR (RT-PCR), nucleic acid sequence-based amplification (NASBA), transcription-based amplification system (TAS), self-sustained sequence replication (3SR), ligation amplification reaction (LAR), Q-beta amplification, and ligase chain reaction (LCR)). Amplicons may comprise RNA or DNA depending on the technique used for amplification. For example, DNA amplicons may be generated by RT-PCR, whereas RNA amplicons may be generated by TAS/NASBA.

The terms “hybridize” and “hybridization” refer to the formation of complexes between nucleotide sequences which are sufficiently complementary to form complexes via Watson-Crick base pairing. Where a primer “hybridizes” with target (template), such complexes (or hybrids) are sufficiently stable to serve the priming function required by, e.g., the DNA polymerase to initiate DNA synthesis.

The terms “selectively detects” or “selectively detecting” refer to the detection of nucleic acids using oligonucleotides (e.g., primers or probes) that are capable of detecting a nucleic acid, for example, by amplifying and/or binding to at least a portion of the nucleic acid, but do not amplify and/or bind to sequences from other nucleic acids under appropriate hybridization conditions.

The “melting temperature” or “Tₘ” of double-stranded DNA is defined as the temperature at which half of the helical structure of DNA is lost due to heating or other dissociation of the hydrogen bonding between base pairs, for example, by acid or alkali treatment, or the like. The Tₘ of a DNA molecule depends on its length and on its base composition. DNA molecules rich in GC base pairs have a higher Tₘ than those having an abundance of AT base pairs. Separated complementary strands of DNA spontaneously reassociate or anneal to form duplex DNA when the temperature is lowered below the Tₘ. The highest rate of nucleic acid hybridization occurs approximately 25 degrees C. below the Tₘ. The Tₘ may be estimated using the following relationship: Tₘ=69.3+0.41(GC) % (Marmur et al. (1962) J. Mol. Biol. 5:109-118).

As used herein, the terms “label” and “detectable label” refer to a molecule capable of detection, including, but not limited to, radioactive isotopes, fluorescers, chemiluminescers, chromophores, enzymes, enzyme substrates, enzyme cofactors, enzyme inhibitors, semiconductor nanoparticles, dyes, metal ions, metal sols, ligands (e.g., biotin, strepavidin or haptens) and the like. The term “fluorescer” refers to a substance or a portion thereof which is capable of exhibiting fluorescence in the detectable range. Particular examples of labels which may be used include, but are not limited to, ethidium bromide, SYBR green, SYBR gold, fluorescein, SYTO-9, SYTO-13, SYTO-16, SYTO-60, SYTO-62, SYTO-64, SYTO-82, PO-PRO-3, YO-PRO-1, SYTOX Orange, and TO-PRO-3, FITC, rhodamine, dansyl, umbelliferone, dimethyl acridinium ester (DMAE), Texas red, luminol, NADPH, horseradish peroxidase (HRP), and α-β-galactosidase. Any of these labels or oligonucleotide primers or probes comprising detectable labels (e.g., TAQMAN probes, molecular beacons, SUNRISE primers, SCORPION primers, and LIGHT-UP probes) can be used for detection of nucleic acids.

A “microfluidic device” is an apparatus or a component of an apparatus that has one or more microfluidic reaction channels and/or chambers. Typically, at least one reaction channel or chamber of a microfluidic device has a cross-sectional dimension between about 0.1 μm and about 1000 μm.

A “separation step” refers to the isolation of an amplified nucleic acid. In certain embodiments, the isolated nucleic acid is used to determine the amount of amplified product or to sequence the amplified product. A “separation step” does not necessarily entail the isolation of all of the amplified product, or that the isolation occurs following a final cycle of the reaction. Instead, a “separation step” can occur at any time during the reaction, and can indicate the isolation of only a fraction of the amplified product.

As used herein, a “biological sample” refers to a sample of tissue or fluid isolated from a subject, including but not limited to, for example, blood, plasma, serum, fecal matter, urine, bone marrow, bile, spinal fluid, lymph fluid, samples of the skin, external secretions of the skin, respiratory, intestinal, and genitourinary tracts, tears, saliva, milk, blood cells, organs, biopsies and also samples of in vitro cell culture constituents including but not limited to conditioned media resulting from the growth of cells and tissues in culture medium, e.g., recombinant cells, and cell components.

### GENERAL

Before describing the devices and methods in detail, it is to be understood that the disclosure is not limited to particular formulations or process parameters as such may, of course, vary. It is also to be understood that the terminology used herein is for the purpose of describing particular embodiments only, and is not intended to be limiting.

Although a number of methods and materials similar or equivalent to those described herein can be used, exemplary preferred materials and methods are described herein.

The present disclosure is based on the discovery of devices and methods for detection and quantification of nucleic acids. Amplification of nucleic acids and separation of amplified products based on size is integrated in a sealed system, which minimizes contamination and eliminates the need for further processing. The nucleic acid amplification reaction is performed in a reaction chamber linked to a separation network by a fluidic connection between the reaction chamber and the separation network. Samples of the amplification reaction mixture can be introduced from the reaction chamber into the separation network by way of this fluidic connection, which allows for multiple sampling, quantitation, and identification of amplicons during the course of an amplification reaction while maintaining a sealed system. The devices and methods described herein increase accuracy of nucleic acid identification and quantification, reduce cost, and permit greater levels of multiplexing. These methods have wide ranging applicability particularly in molecular diagnostics, molecular biology, and forensics.

A more detailed discussion is provided below regarding integrated systems for performing nucleic acid analysis, including microfluidic systems for detection and quantification of nucleic acids, and methods for using such devices, and systems.

### Device for Nucleic Acid Amplification, Size Separation, and Quantification

In one aspect, devices for nucleic acid amplification, size separation and quantification are provided. The devices typically comprise a reaction chamber containing a reaction mixture comprising a sample nucleic acid of interest and reagents for specific nucleic acid amplification, and a fluidic connection between the reaction chamber and a separation network such that a small sample of the reaction mixture can be introduced from the chamber into the separation network. As detailed below, any suitable reaction chamber can be used, including, but not limited to, microtiter plates, glass wells, etc.

The reaction chamber and network are sealed so that nucleic acids cannot be transferred out of the system. The reaction mixture is processed in the chamber such that the nucleic acid is amplified. During the amplification reaction, a sample of the reaction mixture is transferred periodically from the chamber to the separation network where the nucleic acids in the sample are separated based on size. Nucleic acids are detected and quantified after separation, for example, by taking an optical measurement related to the size and concentration of the specific nucleic acids in the sample.

The reaction chamber where nucleic acid amplification takes place is fluidically connected to the separation network. Thus, under normal operating conditions the whole device is sealed and reagents cannot leave the system. In this context, fluidically connected means having a fluid path such that application of some potential difference such as a pressure difference or a voltage difference results in the transfer of nucleic acid from the reaction chamber to the separation network. Separation network means some combination of channels, tubing, wells etc. that can be used to separate nucleic acid fragments based on size. Such separation networks include microfluidic networks, CE capillaries, HPLC columns etc. The reaction chamber is a structure capable of holding the reagents required for the amplification.

The devices described herein allow the formation of amplicons to be measured outside of the reaction chamber while maintaining a sealed system. The particular optical signal used to measure a signal that is a function of the size and concentration of the amplicon can be measured at points before, during and after the amplification reaction.

Sealing the reaction chamber and the separation network allows the risk of contamination to be minimized, allows the detection process to be easily automated and does not require any additional processing outside of the chamber/network device. After addition of the amplification reaction mixture to the reaction chamber and filling of the separation network with an appropriate separation matrix, the device can be sealed to prevent any leakage of reagents from the device. Such sealing can be done for example with a film or membrane. The sealing needs to be done in a manner that the required driving forces can be applied to the separation network and reaction chamber to allow for sampling of the reaction chamber and size separation of the sample. For example, the sealing can be done with a compliant membrane that prevents liquid or aerosols from leaving the device but allows pressure differentials to be applied between the reaction chamber and the separation network. In the case of electrophoretic separations, electrodes can be embedded into the device in such a way that an instrument can control voltages in the device without having to come into contact with the fluid inside the device. Once the measurement has been made, the sealed device can be discarded.

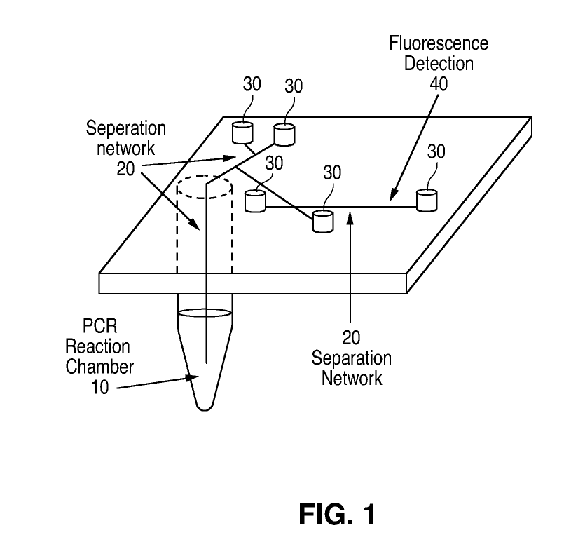

An exemplary device for amplifying nucleic acids by PCR and detecting nucleic acids by measuring fluorescence is depicted in FIG. 1. The PCR reaction chamber 10 comprises a sample nucleic acid and reagents for PCR amplification. Samples are periodically transferred from the reaction chamber through a fluidic connection to the separation network, which comprises a combination of channels 20 and wells 30. The wells 30 provide means to add reagents to the network and apply pressure and electrical gradients. Samples from the amplified reaction mixture can flow into the detection region 40 of the separation network where signals from fluorescently labeled nucleic acids can be detected. It will be appreciated that the drawing is for purposes of illustration only and that nucleic acids can be amplified and detected in a variety of ways.

### Nucleic Acids and Samples of Interest

The nucleic acid of interest to be detected in the methods described herein can be any nucleic acid. The sequences for many nucleic acids and amino acids (from which nucleic acid sequences can be derived via reverse translation) are available. No attempt is made to identify the hundreds of thousands of known nucleic acids, any of which can be detected in the methods provided herein. Common sequence repositories for known nucleic acids include GenBank EMBL, DDBJ and NCBI. Other repositories can easily be identified by searching the internet. The nucleic acid can be RNA (e.g., where amplification could be performed by RT-PCR or LCR) or DNA (e.g., where amplification could be performed by PCR or LCR), or an analogue thereof (e.g., for detection of synthetic nucleic acids or analogues thereof). Any variation in a nucleic acid can be detected, e.g., a mutation, a single nucleotide polymorphism (SNP), an allele, an isotype, a fragment, a full-length nucleic acid, an amplicon, etc. Furthermore, variations in expression levels, fragmentation, or gene copy numbers can be quantitated.

In general, the methods described herein are particularly useful in screening biological samples derived from patients for nucleic acids of interest, e.g., from bodily fluids and/or waste from the patient. Samples derived from relatively large volumes of such materials can be screened (removal of such materials is also relatively non-invasive). The nucleic acids of interest (e.g., present in cancer cells) can easily comprise 1% or less of the related nucleic acid population of the sample (e.g., about 1%, 0.1%, 0.001%, 0.0001% or less of the alleles for a gene of interest). Thus, whole blood, serum, plasma, stool, urine, vaginal secretions, ejaculatory fluid, synovial fluid, a biopsy, cerebrospinal fluid, and amniotic fluid, sputum, saliva, lymph, tears, sweat, or urine, or the like, can easily be screened for rare nucleic acids or fragmentation by the methods described herein, as can essentially any tissue of interest. These samples are typically taken, following informed consent, from a patient by standard medical laboratory methods.

For example, nucleic acids from pathogenic or infectious organisms can be detected, e.g., for infectious fungi, e.g., Aspergillus, or Candida species; bacteria, particularly E. coli, which serves a model for pathogenic bacteria (and, of course certain strains of which are pathogenic), as well as medically important bacteria such as Staphylococci (e.g., aureus), or Streptococci (e.g., pneumoniae); protozoa such as sporozoa (e.g., Plasmodia), rhizopods (e.g., Entamoeba) and flagellates (Trypanosoma, Leishmania, Trichomonas, Giardia, etc.); viruses such as (+) RNA viruses (examples include Poxviruses e.g., vaccinia; Picornaviruses, e.g. polio; Togaviruses, e.g., rubella; Flaviviruses, e.g., HCV; and Coronaviruses), (−) RNA viruses (e.g., Rhabdoviruses, e.g., VSV; Paramyxovimses, e.g., RSV; Orthomyxovimses, e.g., influenza; Bunyaviruses; and Arenaviruses), dsDNA viruses (Reoviruses, for example), RNA to DNA viruses, i.e., Retroviruses, e.g., HIV and HTLV, and certain DNA to RNA viruses such as Hepatitis B. Single and low copy amplification methods described herein can be useful in many cases, e.g., in exudates from bacterial infections to identify living (having full length nucleic acids) versus dead and lysed pathogens (having fragmented nucleic acids).

### Nucleic Acid Amplification

Also described herein are methods of amplifying one or more sequences of a nucleic acid of interest from a sample or aliquot and, optionally, one or more additional nucleic acids. Any available amplification method can be used, including PCR, RT-PCR, NASBA, TAS, 3SR, LAR, Q-beta, LCR, or any other method of nucleic acid amplification. PCR, RT-PCR, and LCR are preferred amplification methods for amplifying a nucleic acid of interest. Real time PCR and/or RT-PCR (e.g., mediated via TAQMAN probes or molecular beacon-based probes) can also be used to facilitate detection of amplified nucleic acids.

It is expected that one of skill is generally familiar with the details of these amplification methods. Details regarding these amplification methods can be found, e.g., in Sambrook (supra); Ausubel (supra); Innis (supra); EPA 684,315; EPA 320,308; EPA 439,182; WO 93/22461; PCR: A Practical Approach (The Practical Approach Series) by Quirke et al. (eds.). (1992) by Oxford University Press. Mullis et al., (1987) U.S. Pat. No. 4,683,202; PCR Protocols A Guide to Methods and Applications (Innis et al. eds) Academic Press Inc. San Diego, Calif. (1990) (Innis); Arnheim & Levinson (Oct. 1, 1990) C&EN 36-47; The Journal Of NIH Research (1991) 3, 81-94; (Kwoh et al. (1989) Proc. Natl. Acad. Sci. USA 86, 1173; Guatelli et al. (1990) Proc. Natl. Acad. Sci. USA 87:1874-1878; Compton (1991) Nature 350:91-92 (1991), Walker et al. (1996) Clin. Chem. 42:9-13; Lomell et al. (1989) J. Clin. Chem 35, 1826; Landegren et al, (1988) Science 241, 1077-1080; Van Brunt (1990) Biotechnology 8, 291-294; Wu and Wallace, (1989) Gene 4, 560; Barringer et al. (1990) Gene 89, 117, Sooknanan and Malek (1995) Biotechnology 13: 563-564; Hill (2001) Expert Rev. Mol. Diagn. 1:445-55; WO 89/1050; WO 88/10315; EPO Publication No. 408,295; EPO Application No. 8811394-8.9; WO91/02818; U.S. Pat. Nos. 5,399,491, 6,686,156, and 5,556,771; herein incorporated by reference in their entireties. Improved methods of cloning in vitro amplified nucleic acids are described in Wallace et al., U.S. Pat. No. 5,426,039. Improved methods of amplifying large nucleic acids by PCR are summarized in Cheng et al. (1994) Nature 369: 684-685 and the references therein, in which PCR amplicons of up to 40 kb are generated.

Additional details can also be found in the literature for a variety of applications of PCR. For example, details regarding amplification of nucleic acids in plants can be found, e.g., in Plant Molecular Biology (1993) Croy (ed.) BIOS Scientific Publishers, Inc. Similarly, additional details regarding PCR for cancer detection can be found in any of a variety of sources, e.g., Bernard and Wittwer (2002) “Real Time PCR Technology for Cancer Diagnostics Clinical Chemistry 48(8): 1178-1185; Perou et al. (2000) “Molecular portraits of human breast tumors” Nature 406:747-52; van't Veer et al. (2002) “Gene expression profiling predicts clinical outcome of breast cancer” Nature 415:530-6; Rosenwald et al. (2001) “Relation of gene expression phenotype to immunoglobulin mutation genotype in B cell chronic lymphocytic leukemia” J Exp Med 194: 1639-47; Alizadeh et al. (2000) “Distinct types of diffuse large B-cell lymphoma identified by gene expression profiling” Nature 403:503-11; Garber et al. (2001) “Diversity of gene expression in adenocarcinoma of the lung” Proc Natl Acad Sci USA 98:13784-9; Tirkkonen et al. (1998) “Molecular cytogenetics of primary breast cancer by CGH” Genes Chromosomes Cancer 21:177-84; Watanabe et al. (2001) “A novel amplification at 17q21-23 in ovarian cancer cell lines detected by comparative genomic hybridization” Gynecol Oncol 81:172-7, and many others.

### General Probe Synthesis Methods

In general, synthetic methods for making oligonucleotides, including probes, molecular beacons, PNAs, LNAs (locked nucleic acids), etc., are well known. For example, oligonucleotides can be synthesized chemically according to the solid phase phosphoramidite triester method described by Beaucage and Caruthers (1981), Tetrahedron Letts., 22(20):1859-1862, e.g., using a commercially available automated synthesizer, e.g., as described in Needham-VanDevanter et al. (1984) Nucleic Acids Res., 12:6159-6168. Oligonucleotides, including modified oligonucleotides can also be ordered from a variety of commercial sources known to persons of skill. There are many commercial providers of oligo synthesis services, and thus this is a broadly accessible technology. Any nucleic acid can be custom ordered from any of a variety of commercial sources, such as The Midland Certified Reagent Company (mcrc@oligos.com), The Great American Gene Company (genco.com), ExpressGen Inc. (expressgen.com), Operon Technologies Inc. (Alameda, Calif.) and many others. Similarly, PNAs can be custom ordered from any of a variety of sources, such as PeptidoGenic (pkim@ccnet.com), HTI Bio-products, inc. (htibio.com), BMA Biomedicals Ltd (U.K.), Bio-Synthesis, Inc., and many others.

### Amplification in Microfluidic Systems

A number of high throughput approaches to performing PCR and other amplification reactions have been developed, e.g., involving amplification reactions in microfluidic devices, as well as methods for detecting and analyzing amplified nucleic acids in or on the devices. Details regarding such technology is found, e.g., in the technical and patent literature, e.g., Kopp et al. (1998) “Chemical Amplification: Continuous Flow PCR on a Chip” Science, 280 (5366):1046; U.S. Pat. No. 6,444,461 to Knapp, et al. (Sep. 3, 2002) MICROFLUIDIC DEVICES AND METHODS FOR SEPARATION; U.S. Pat. No. 6,406,893 to Knapp, et al. (Jun. 18, 2002) MICROFLUIDIC METHODS FOR NON-THERMAL NUCLEIC ACID MANIPULATIONS; U.S. Pat. No. 6,391,622 to Knapp, et al. (May 21, 2002) CLOSED-LOOP BIOCHEMICAL ANALYZERS; U.S. Pat. No. 6,303,343 to Kopf-Sill (Oct. 16, 2001) INEFFICIENT FAST PCR; U.S. Pat. No. 6,171,850 to Nagle, et al. (Jan. 9, 2001) INTEGRATED DEVICES AND SYSTEMS FOR PERFORMING TEMPERATURE CONTROLLED REACTIONS AND ANALYSES; U.S. Pat. No. 5,939,291 to Loewy, et al. (Aug. 17, 1999) MICROFLUIDIC METHOD FOR NUCLEIC ACID AMPLIFICATION; U.S. Pat. No. 5,955,029 to Wilding, et al. (Sep. 21, 1999) MESOSCALE POLYNUCLEOTIDE AMPLIFICATION DEVICE AND METHOD; U.S. Pat. No. 5,965,410 to Chow, et al. (Oct. 12, 1999) ELECTRICAL CURRENT FOR CONTROLLING FLUID PARAMETERS IN MICROCHANNELS; Service (1998) “Microchips Arrays Put DNA on the Spot” Science 282:396-399), Zhang et al. (1999) “Automated and Integrated System for High-Throughput DNA Genotyping Directly from Blood” Anal. Chem. 71:1138-1145 and many others.

For example, U.S. Pat. No. 6,391,622 to Knapp, et al. (May 21, 2002) CLOSED-LOOP BIOCHEMICAL ANALYZERS and the references cited therein describes systems comprising microfluidic elements that can access reagent storage systems and that can perform PCR or other amplification reactions by any of a variety of methods in the microfluidic system.

Alternatively, PCR amplicons can be detected by conventional methods, such as hybridization to a labeled probe, e.g., prior to or following a separation operation that separates unhybridized probe from hybridized probe. For example, an electrophoretic separation can be performed in a channel of the microscale device.

### Separation Network

The products of the nucleic acid amplification reaction are separated in the separation network (e.g., by electrophoresis or flowing through a sieving matrix). A wide variety of sieving and molecular partition matrixes are available, and can be used in the separation network. For example, a variety of sieving matrixes, partition matrixes and the like are available from Supelco, Inc. (Bellefonte, Pa.; see, 1997 Suppleco catalogue). Common matrixes which are useful in the devices and methods described herein include those generally used in low pressure liquid chromatography, gel electrophoresis and other liquid phase separations; matrix materials designed primarily for non-liquid phase chromatography are also useful in certain contexts, as the materials often retain separatory characteristics when suspended in fluids. Sieving matrixes typically include one or more of the following polymers: acrylamide, agarose, methyl cellulose, polyethylene oxide, hydroxycellulose, hydroxy ethyl cellulose, or the like. Combinations of any of these polymers are also optionally used. Various types of acrylamide are used, including, but not limited to, linear acrylamide, polyacrylamide, polydimethylacrylamide, polydimethylacrylamide/coacrylic acid, or the like. For a discussion of electrophoresis see, e.g., Weiss (1995) Ion Chromatography VCH Publishers Inc.; Baker (1995) Capillary Electrophoresis John Wiley and Sons; Kuhn (1993) Capillary Electrophoresis: Principles and Practice Springer Verlag; Righetti (1996) Capillary Electrophoresis in Analytical Biotechnology CRC Press; Hill (1992) Detectors for Capillary Chromatography John Wiley and Sons; Gel Filtration: Principles and Methods (5th Edition) Pharmacia; Gooding and Regnier (1990) HPLC of Biological Macromolecules: Methods and Applications (Chrom. Sci. Series, volume 51) Marcel Dekker and Scott (1995) Techniques and Practices of Chromatography Marcel Dekker, Inc.

Commercially available low pressure liquid chromatography media include, e.g., non-ionic macroreticular and macroporous resins which adsorb and release components based upon hydrophilic or hydrophobic interactions such as Amberchrom resins (highly cross-linked styrene/divinylbenzene copolymers suitable for separation of peptides, proteins, nucleic acids, antibiotics, phytopharmacologicals, and vitamins); the related Amberlite XAD series resins (polyaromatics and acrylic esters) and amberchroms (polyaromatic and polymethacrylates) (manufactured by Rohm and Haas, available through Suppleco); Diaion (polyaromatic or polymethacrylic beads); Dowex (polyaromatics or substituted hydrophilic functionalized polyaromatics) (manufactured by Dow Chemical, available through Suppleco); Duolite (phenol-formaldehyde with methanolic functionality). MCI GEL,sephabeads, supelite DAX-8 (acrylic ester) and Supplepak (polyaromatic) (all of the preceding materials are available from Suppleco). For a description of uses for Amberlite and Duolite resins, see, Amberlite/Duolite Anion Exchange Resins (Available from Supplecoe, Cat No. T412141). Gel filtration chromatography matrixes are also suitable, including sephacryl, sephadex, sepharose, superdex, superose, toyopearl, agarose, cellulose, dextrans, mixed bead resins, polystyrene, nuclear resins, DEAE cellulose, Benzyl DEA cellulose, TEAE cellulose, and the like (Suppleco).

Gel electrophoresis media include silica gels such as Davisil Silica, E. Merck Silica Gel, Sigma-Aldrich Silica Gel (all available from Suppleco) in addition to a wide range of silica gels available for various purposes as described in the Aldrich catalogue/handbook (Aldrich Chemical Company (Milwaukee, Wis.)). Preferred gel materials include agarose based gels, various forms of acrylamide based gels (reagents available from, e.g., Suppleco, SIGMA, Aldrich, SIGMA-Aldrich and many other sources) colloidial solutions such as protein colloids (gelatins) and hydrated starches. Various forms of gels are discussed further below.

A variety of affinity media for purification and separation of molecular components are also available, including a variety of modified silica gels available from SIGMA, Aldrich and SIGMA-Aldrich, as well as Suppleco, such as acrylic beads, agarose beads, cellulose, sepharose, sepharose CL, toyopearl or the like chemically linked to an affinity ligand such as a biological molecule. A wide variety of activated matrixes, amino acid resins, avidin and biotin resins, carbohydrate resins, dye resins, glutathione resins, hydrophobic resins, immunochemical resins, lectin resins, nucleotide/coenzyme resins, nucleic acid resins, and specialty resins are available, e.g., from Suppleco, SIGMA, Aldrich or the like. See also, Hermanson et al. (1992) Immobilized Affinity Ligand Techniques Academic Press.

Other media commonly used in chromatography are also adaptable to the present disclosure, including activated aluminas, carbopacks, carbosieves, carbowaxes, chromosils, DEGS, Dexsil, Durapak, Molecular Sieve, OV phases, pourous silica, chromosorb series packs, HayeSep series, Porapak series, SE-30, Silica Gel, SP-1000, SP-1200, SP-2100, SP-2250, SP-2300, SP2401, Tenax, TCEP, supelcosil LC-18-S and LC-18-T, Methacrylate/DVBm, polyvinylalcohols, napthylureas, non-polar methyl silicone, methylpolysiloxane, poly (ethylene glycol) biscyanopropyl polysiloxane and the like.

In certain embodiments, the integrated system comprises a microfluidic device. Several methods of providing fluidic regions in selected regions of a channel, or selected channels of a microfluidic device are provided. In a first aspect, multiple microfluidic regions are filled with a first fluid such as an unpolymerized solution that, upon polymerization, forms a sieving matrix. Elements of the microfluidic device such as microfluidic channels are filled with the first fluid by forcing the fluid into the channel under pressure, or by moving the fluid into the channel electrokinetically.

In another embodiment, the first fluid is polymerized by selectively exposing certain channel regions to an activator or cross-linker. For example, where the fluid is polyacrylamide, the activator/cross linker can be TEMED and APS. In this embodiment, the reagents are placed into a well and electrokinetically loaded into selected channel regions of a microfluidic substrate. After selective exposure to activator/cross linker as appropriate, unpolymerized materials are removed from regions where monomer material is undesirable, typically using electroosmotic flow (but optionally using a pressure gradient). Often the material will be shunted to one or more waste buffer where the material is optionally removed, e.g., by pipetting or electropipeting the material out of the well.

In another embodiment, a sieving matrix is deposited throughout a channel or channels of a microfluidic device in a form which is subject to electroosmosis (i.e., the matrix moves electrokinetically in the channel). The matrix is then selectively replaced by a second fluidic phase (e.g., a buffer) in selected regions of a channel by electrokinetically loading the buffer in the selected region.

In an additional embodiment, a first fluidic phase is loaded into multiple channels of a microfluidic device and polymerized in place. Selective components which solubilize the polymerized gel are then loaded (e.g., electrokinetically or under pressure) into channel regions where polymerized product is not desired. The selected components dissolve the polymerized gel. Example of solubilization compounds include acids, bases and other chemicals. In one preferred embodiment, at least two compounds are used to dissolve polymerized products, where both products need to be present to dissolve the polymer. This provides for fine control of dissolution, e.g., where each chemical is under separate electrokinetic control. An example of such a chemical pair is DTT(N,N′-bis(acrylol)cystamine or (1,2-dihydroxyethylene-bis-acrylamide) [DHEBA] and sodium periodiate or calcium alginate+EDTA or TCEP-HCL and N,N′-bis(acryloyl)cystamine. A variety of such materials are known.

### Detecting the Amplified Nucleic Acids

Many available methods for detecting amplified nucleic acids can be used in the devices and methods of the present disclosure. Common approaches include detection of intercalating dyes (e.g., ethidium bromide or SYBR green), detection of labels incorporated into the amplification probes or the amplified nucleic acids themselves, e.g., following electrophoretic separation of the amplification products from unincorporated label), and/or detection of secondary reagents that bind to the nucleic acids. Details of these general approaches are found in the references cited herein, e.g., Sambrook (2000), Ausubel (2002), and the references in the sections herein related to real time PCR detection. Additional labeling strategies for labeling nucleic acids and corresponding detection strategies can be found, e.g., in Haugland (1996) Handbook of Fluorescent Probes and Research Chemicals Sixth Edition by Molecular Probes, Inc. (Eugene Oreg.); or Haugland (2001) Handbook of Fluorescent Probes and Research Chemicals Eighth Edition by Molecular Probes, Inc. (Eugene Oreg.) (Available on CD ROM).

Amplified nucleic acids (amplicons) can be detected during or after separation (e.g., by electrophoresis). In a preferred embodiment, nucleic acids are detected after separation. Inclusion of reagents for detection of nucleic acids within the separation network, rather than the reaction chamber, has the advantage of not requiring optimization of the amplification reaction in the presence of the reagents used for detection. Separation of the amplification and detection functions allows for optimization of reaction and detection conditions independently.

Available microfluidic systems that include detection features for detecting nucleic acids include the LabChip 90 SE from Caliper Life Sciences, Inc. (Mountain View, Calif.), as well as the Agilent 2100 bioanalyzer (Agilent, Palo Alto, Calif.). Additional details regarding systems that comprise detection capabilities are well described in the patent literature, e.g., the references already noted herein and in Parce et al. “High Throughput Screening Assay Systems in Microscale Fluidic Devices” WO 98/00231.

In general, the devices herein optionally include signal detectors, e.g., which detect fluorescence, phosphorescence, radioactivity, pH, charge, absorbance, luminescence, temperature, magnetism or the like. Fluorescent detection is especially preferred and generally used for detection of amplified nucleic acids (however, upstream and/or downstream operations can be performed on amplicons, which can involve other detection methods, such as mass spectroscopy or size exclusion).

The detector(s) optionally monitor one or a plurality of signals from an amplification reaction and/or hybridization reaction. For example, the detector can monitor optical signals which correspond to “real time” amplification assay results. The detector can monitor a single type of signal, or, e.g., simultaneously monitor multiple different signals.

Example detectors include photo multiplier tubes, spectrophotometers, CCD arrays, scanning detectors, microscopes, galvo-scanns and/or the like. Amplicons or other components which emit a detectable signal can be flowed past the detector, or, alternatively, the detector can move relative to the site of the amplification reaction (or, the detector can simultaneously monitor a number of spatial positions corresponding to channel regions, or microtiter wells e.g., as in a CCD array). Detectors can detect signals from probes associated with nucleic acids that flow into one or more detection regions, e.g., of a microfluidic device.

The detector can include or be operably linked to a computer (or other logic device), e.g., which has software for converting detector signal information into assay result information (e.g., presence of a nucleic acid of interest, the length of a nucleic acid of interest, proportions of nucleic acid of interest lengths, and/or correlations with disease states), or the like.

Particularly preferred detection systems include optical detection systems for detecting an optical property of a material within the channels and/or chambers of the microfluidic devices that are incorporated into the microfluidic systems described herein. Such optical detection systems are typically placed adjacent to a microscale channel of a microfluidic device, and are in sensory communication with the channel via an optical detection window that is disposed across the channel or chamber of the device. Optical detection systems include systems that are capable of measuring the light emitted from material within the channel, the transmissivity or absorbance of the material, as well as the materials spectral characteristics. In preferred aspects, the detector measures an amount of light emitted from the material, such as a fluorescent or chemiluminescent material. As such, the detection system will typically include collection optics for gathering a light based signal transmitted through the detection window, and transmitting that signal to an appropriate light detector. Microscope objectives of varying power, field diameter, and focal length are readily utilized as at least a portion of this optical train. The light detectors are optionally spectrophotometers, photodiodes, avalanche photodiodes, photomultiplier tubes, diode arrays, or in some cases, imaging systems, such as charged coupled devices (CCDs) and the like. The detection system is typically coupled to a computer, via an analog to digital or digital to analog converter, for transmitting detected light data to the computer for analysis, storage and data manipulation.

In the case of fluorescent materials such as labeled amplicons, the detector typically includes a light source that produces light at an appropriate wavelength for activating the fluorescent material, as well as optics for directing the light source through the detection window to the material contained in the channel or chamber. The light source can be any number of light sources that provides an appropriate wavelength, including lasers, laser diodes, and LEDs. Other light sources are used in other detection systems. For example, broad band light sources are typically used in light scattering/transmissivity detection schemes, and the like. Typically, light selection parameters are well known to those of skill in the art.

### Additional System Details

The systems described herein can include microfluidic devices, reaction mixtures, detectors, sample storage elements (microtiter plates, dried arrays of components, etc.), flow controllers, amplification devices or microfluidic modules, computers and/or the like. These systems can be used for aliquoting, amplifying and analyzing the nucleic acids of interest. The microfluidic devices, amplification components, detectors and storage elements of the systems have already been described in some detail above. The following discussion describes appropriate controllers and computers, though many configurations are available and one of skill would be expected to be familiar in their use and would understand how they can be applied to the present disclosure.

### Flow Controllers

A variety of controlling instrumentation is optionally utilized in conjunction with the microfluidic devices described herein, for controlling the transport and direction of fluids and/or materials within the devices described herein, e.g., by pressure-based or electrokinetic control.

For example, in many cases, fluid transport and direction are controlled in whole or in part, using pressure based flow systems that incorporate external or internal pressure sources to drive fluid flow. Internal sources include microfabricated pumps, e.g., diaphragm pumps, thermal pumps, Lamb wave pumps and the like that have been described in the art. See, e.g., U.S. Pat. Nos. 5,271,724, 5,277,556, and 5,375,979 and Published PCT Application Nos. WO 94/05414 and WO 97/02357. The systems described herein can also utilize electrokinetic material direction and transport systems.

Preferably, external pressure sources are used, and applied to ports at channel termini. These applied pressures, or vacuums, generate pressure differentials across the lengths of channels to drive fluid flow through them. In the interconnected channel networks described herein, differential flow rates on volumes are optionally accomplished by applying different pressures or vacuums at multiple ports, or preferably, by applying a single vacuum at a common waste port and configuring the various channels with appropriate resistance to yield desired flow rates. Example systems are described in U.S. Published Application No. 2002-0019059 published on Feb. 14, 2002.

Typically, the controller systems are appropriately configured to receive or interface with a microfluidic device or system element as described herein. For example, the controller and/or detector, optionally includes a stage upon which a microfluidic device is mounted to facilitate appropriate interfacing between the controller and/or detector and the device. Typically, the stage includes an appropriate mounting/alignment structural element, such as a nesting well, alignment pins and/or holes, asymmetric edge structures (to facilitate proper device alignment), and the like. Many such configurations are described in the references cited herein.

The controlling instrumentation discussed above is also optionally used to provide for electrokinetic injection or withdrawal of material downstream of the region of interest to control an upstream flow rate. The same instrumentation and techniques described above are also utilized to inject a fluid into a downstream port to function as a flow control element.

### Computer

As noted above, either or both of the controller system and/or the detection system can be coupled to an appropriately programmed processor or computer (logic device) which functions to instruct the operation of these instruments in accordance with preprogrammed or user input instructions, receive data and information from these instruments, and interpret, manipulate and report this information to the user. As such, the computer is typically appropriately coupled to one or both of these instruments (e.g., including an analog to digital or digital to analog converter as needed).

The computer typically includes appropriate software for receiving user instructions, either in the form of user input into a set parameter fields, e.g., in a GUI, or in the form of preprogrammed instructions, e.g., preprogrammed for a variety of different specific operations. The software then converts these instructions to appropriate language for instructing the operation of the fluid direction and transport controller to carry out the desired operation. The computer then receives the data from the one or more sensors/detectors included within the system, and interprets the data, either provides it in a user understood format, or uses that data to initiate further controller instructions, in accordance with the programming, e.g., such as in monitoring and control of flow rates (including for continuous flow), temperatures, applied voltages, and the like.

The systems and/or kits can include system instructions (e.g., embodied in a computer or in a computer readable medium, e.g., as system software) for practicing any of the method steps herein. For example, the system optionally includes system software that correlates a shape, length, width, volume and/or area occupied by amplified copies of the nucleic acid of interest, as detected by the detector, to the number of copies of the nucleic acid of interest present in one of the aliquots, or to the number of copies of the nucleic acid of interest present in the sample, or both. Similarly, the system optionally includes system instructions that direct the dilution module to aliquot the sample into a plurality of aliquots, including a plurality of zero copy aliquots comprising no copies of the nucleic acids of interest and one or more single copy aliquot comprising a single copy of the nucleic acid of interest.

The statistical functions noted above can also be incorporated into system software, e.g., embodied in the computer, in computer memory or on computer readable media. For example, the computer can include statistical or probabilistic system software that performs one or more statistical or probabilistic analysis of signals received from one or more of the aliquots subjected to amplification (e.g., via thermocycling). For example, the statistical or probabilistic analysis can include Poisson analysis, Monte Carlo analysis, application of a genetic algorithm, neural network training, Markov modeling, hidden Markov modeling, multidimensional scaling, PLS analysis, and/or PCA analysis. The statistical or probabilistic analysis software optionally quantitatively determines a concentration, proportion, or number of the nucleic acids of interest in the sample.

Computers and software of the systems receive and evaluate signal data from one or more analyses to provide quantitation and/or proportionality determinations for nucleic acids of interest. In a basic form, e.g., the amplitude or integrated area of a signal can be adjusted with a conversion factor for an output in desired units, such as, e.g., copies per nL, ng/μL, and the like. Alternately, one or more standard materials of known concentration can be analyzed to provide data for regression analyses wherein changes in detectable signals with changes in concentration are expressed as an equation (standard curve) from which unknown concentrations can be determined by insertion of one or more signal parameters into the equation. In a particular embodiment, quantitation of a nucleic acid of interest can be based on the number of amplification cycles required to obtain a signal of a certain intensity.

Typically, the computer includes software for the monitoring of materials in the channels. Additionally, the software is optionally used to control electrokinetic or pressure modulated injection or withdrawal of material. The injection or withdrawal is used to modulate the flow rate as described above, to mix components, and the like.

### Kits

The present invention also provides kits for carrying out the methods described herein. In particular, these kits typically include system components described herein, as well as additional components to facilitate the performance of the methods by an investigator.

The kit also typically includes a receptacle in which the system component is packaged. The elements of the kits of the present invention are typically packaged together in a single package or set of related packages. The package optionally includes reagents used for amplification, detection, and/or quantification of nucleic acids, e.g., buffers, amplification reagents, probes, dyes or other detection reagents, standard reagents, and the like, and a membrane, film or other agent for sealing the system, as well as written instructions for carrying out the methods described herein. In the case of prepackaged reagents, the kits optionally include pre-measured or pre-dosed reagents that are ready to incorporate into the methods without measurement, e.g., pre-measured fluid aliquots, or pre-weighed or pre-measured solid reagents that may be easily reconstituted by the end-user of the kit.

Generally, the microfluidic devices described herein are optionally packaged to include reagents for performing the device's preferred function. For example, the kits can include any of microfluidic devices described along with assay components, reagents, sample materials, control materials, or the like. Such kits also typically include appropriate instructions for using the devices and reagents, and in cases where reagents are not predisposed in the devices themselves, with appropriate instructions for introducing the reagents into the channels and/or chambers of the device. In this latter case, these kits optionally include special ancillary devices for introducing materials into the microfluidic systems, e.g., appropriately configured syringes/pumps, or the like (in one preferred embodiment, the device itself comprises a pipettor element, such as an electropipettor for introducing material into channels and chambers within the device). In the former case, such kits typically include a microfluidic device with necessary reagents predisposed in the channels/chambers of the device. Generally, such reagents are provided in a stabilized form, so as to prevent degradation or other loss during prolonged storage, e.g., from leakage. A number of stabilizing processes are widely used for reagents that are to be stored, such as the inclusion of chemical stabilizers (i.e., enzymatic inhibitors, microcides/bacteriostats, anticoagulants), the physical stabilization of the material, e.g., through immobilization on a solid support, entrapment in a matrix (i.e., a gel), lyophilization, or the like.

### Experimental

Below are examples of specific embodiments for carrying out the present disclosure. The examples are offered for illustrative purposes only, and are not intended to limit the scope of the present disclosure in any way.

Efforts have been made to ensure accuracy with respect to numbers used (e.g., amounts, temperatures, etc.), but some experimental error and deviation should, of course, be allowed for.

### EXAMPLE 1

### Device for PCR Amplification and Quantification of Nucleic Acids

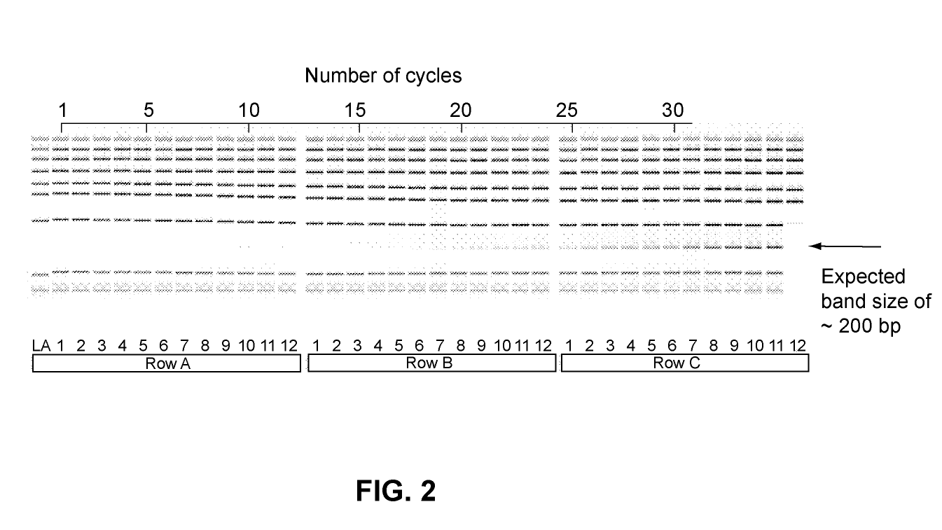

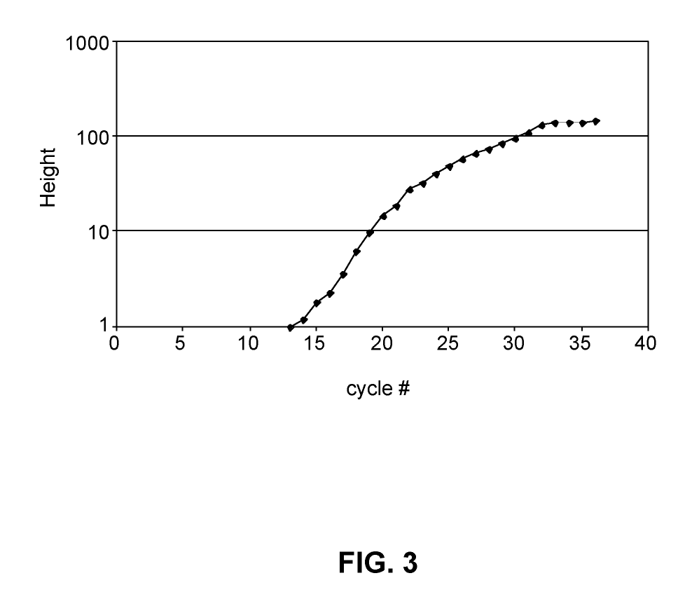

PCR was performed in a standard PCR tube in a thermocycler (MJ Research). As shown schematically in FIG. 1, a LabChip 90 microfluidics system (Caliper Life Sciences) with a DNA 5K chip was placed over the thermocycler such that the sipper was placed in 35 μl of the PCR reaction overlayed with 15 μl of oil. A plastic sheath was glued around the sipper and a plastic tube was press fit between the sheath and the PCR well in order to seal the chip to the PCR well. The thermocycling protocol and sipping protocol were synchronized so that a sample was taken from the PCR reaction and analyzed at approximately the same time during each PCR thermocycle. As shown in FIGS. 2 and 3, the amount of amplicon was quantified during each cycle. Standard Count analysis can be used with a reference curve to quantify the amount of nucleic acid in the starting material.

Thus, methods are described for detecting and quantifying nucleic acids using a sealed system that minimizes contamination. The method permits multiple sampling of an amplification reaction mixture and separation and identification of nucleic acids. Although preferred embodiments have been described in some detail, it is understood that obvious variations can be made without departing from the spirit and the scope of the disclosure.

1. A method for detecting and quantifying one or more specific nucleic acids in a sealed system, the method comprising:
a) providing a device comprising:
i) a reaction chamber containing a reaction mixture comprising one or more nucleic acids and reagents for specific nucleic acid amplification,
ii) a separation network, and
iii) a fluidic connection between the reaction chamber and the separation network, wherein an aliquot of the reaction mixture can be removed from the chamber into the separation network;
b) sealing the reaction chamber and network so that the nucleic acids cannot be transferred out of the device;
c) processing the reaction mixture in the reaction chamber whereby one or more nucleic acids are amplified,
d) periodically transferring an aliquot of the reaction mixture from the reaction chamber to the separation network during the amplification reaction of the nucleic acids;
e) separating the nucleic acids in the separation network based on the size of the nucleic acids; and
f) detecting the separated nucleic acids.

2. The method of claim 1, wherein the nucleic acids are amplified by a method selected from the group consisting of polymerase chain reaction (PCR), reverse-transcriptase PCR (RT-PCR), nucleic acid sequence-based amplification (NASBA), transcription-based amplification system (TAS), self-sustained sequence replication (3SR), ligation amplification reaction (LAR), Q-beta amplification, and ligase chain reaction (LCR).

3. The method of claim 1, wherein samples are withdrawn from the reaction chamber after each cycle of nucleic acid amplification by applying a pressure between the reaction chamber and the separation network.

4. The method of claim 1, wherein more than one nucleic acid is analyzed.

5. The method of claim 4, wherein at least 7 nucleic acid templates are analyzed.

6. The method of claim 1, wherein the separation network comprises channels, tubing, or wells that can be used to separate nucleic acids or fragments thereof based on size.

7. The method of claim 6, wherein the separation network comprises a microfluidic network.

8. The method of claim 1, wherein the nucleic acids are separated electrophoretically.

9. The method of claim 8, wherein the separation network comprises a CE capillary.

10. The method of claim 1, wherein the nucleic acids are separated chromatographically.

11. The method of claim 10, wherein the separation network comprises an HPLC column.

12. The method of claim 1, wherein the nucleic acids are separated by flowing said nucleic acids through a sieving matrix.

13. The method of claim 12, wherein the separation network comprises a sieving matrix comprising a polymer selected from the group consisting of linear acrylamide, polyacrylamide, polydimethylacrylamide, polydimethylacrylamide/coacrylic acid, agarose, methyl cellulose, polyethylene oxide, hydroxycellulose, and hydroxy ethyl cellulose.

14. The method of claim 1, wherein the reaction chamber is sealed with a film.

15. The method of claim 1, wherein the reaction chamber is sealed with a membrane.

16. The method of claim 15, wherein the membrane is a compliant membrane that prevents liquid or aerosols from leaving the device, but allows pressure differentials to be applied between the reaction chamber and the separation network.

17. The method of claim 1, wherein the separation network comprises an electrophoresis device.

18. The method of claim 17, wherein the system comprises a microfluidic device for gel electrophoresis.

19. The method of claim 1, wherein the nucleic acids are detected with a detector comprising a fluorometer, a charge coupled device, a laser, an enzyme, an enzyme substrate, a photo multiplier tube, a spectrophotometer, scanning detector, microscope, or a galvo-scanner.

20. The method of claim 19, wherein the nucleic acids are detected by measuring absorbance.

21. The method of claim 19, wherein the nucleic acids are detected by measuring fluorescence.

22. The method of claim 1, wherein the nucleic acids are detected by measuring one or more signals from one or more detectably labeled probes that selectively bind to the nucleic acids.

23. The method of claim 1, wherein the nucleic acids are detected by measuring one or more signals from one or more detectably labeled primers incorporated into the nucleic acids during amplification.

24. The method of claim 1, wherein the nucleic acids are detecting by measuring the signal from an intercalating dye.

25. The method of claim 24, wherein the intercalating dye is ethidium bromide or SYBR green.

26. The method of claim 1, wherein reagents for detection of nucleic acids are added to the separation network.

27. The method of claim 1, wherein nucleic acids are detected by measurement of fluorescence from an intercalating dye in the separation network.

28. The method of claim 1, wherein a nucleic acid of interest in the sample is quantified by amplifying the nucleic acid of interest through a plurality of amplification cycles; detecting signals associated with amplicons produced for two or more of the amplification cycles; preparing a sample curve of a signal parameter versus a number of amplification cycles; and, comparing one or more identifiable points from the sample curve to a standard curve of identifiable points versus concentration, thereby quantifying the nucleic acid of interest.

29. The method of claim 1, wherein the nucleic acids are amplified by PCR.

30. The method of claim 29, wherein samples are withdrawn from the reaction chamber during each thermocycle by applying a pressure between the reaction chamber and the separation network, separation of nucleic acids is based on microfluidic gel electrophoresis, and detection of nucleic acids is accomplished by fluorescence of an intercalating dye introduced in the separation network.

31. The method of claim 30, wherein the intercalating dye is ethidium bromide.

32. The method of claim 30, wherein the intercalating dye is SYBR green.

33-43. (canceled)
